(19) United States
(12) Patent Application Publication
(10) Pub. No.: US 2024/0185968 A1
(43) Pub. Date: Jun. 6, 2024

(54) A Dialogue-Based Medical Decision System

(71) Applicant: BIOTRONIK SE & Co. KG, Berlin (DE)

(72) Inventors: Thomas DOERR, Berlin (DE); Jens MUELLER, Berlin (DE); Matthias GRATZ, Erlangen (DE); R. Hollis WHITTINGTON, Portland, OR (US); Miro SELENT, Nuthetal (DE)

(73) Assignee: BIOTRONIK SE & Co. KG, Berlin (DE)

(21) Appl. No.: 18/553,663

(22) PCT Filed: Apr. 12, 2022
(86) PCT No.: PCT/EP2022/059736

§ 371 Date: Oct. 2, 2023

Related U.S. Application Data
(60) Provisional application No. 63/176,917, filed on Apr. 20, 2021.

(30) Foreign Application Priority Data
Jun. 8, 2021 (EP) 21178109.1

(51) Int. Cl. G16H 10/20; G16H 10/60; G16H 40/67; G16H 50/20; G16H 80/00

(52) U.S. Cl. CPC G16H 10/20; G16H 10/60; G16H 40/67; G16H 50/20; G16H 80/00

(57) ABSTRACT

The present disclosure relates to an apparatus and a method for ensuring patient compliance during a recovery process. In particular, the present disclosure relates to a dialogue-based medical decision system comprising a receiver for receiving data associated with a health state of the patient sent from a device associated with the patient and a processing unit adapted to select a message associated with the health state of the patient based on the received data and based on stored patient data. Moreover, the dialogue-based medical decision system comprises a transmitter for transmitting the message to the device and/or another device associated with the patient.

### CROSS-REFERENCE TO RELATED APPLICATIONS

This application is the United States National Phase under 35 U.S.C. § 371 of PCT International Patent Application No. PCT/EP2022/059736, filed on Apr. 12, 2022, which claims the benefit of European Patent Application No. 21178109.1, filed on Jun. 8, 2021, and U.S. Provisional Patent Application No. 63/176,917, filed on Apr. 20, 2021, the disclosures of which are hereby incorporated by reference herein in their entireties.

### TECHNICAL FIELD

The present disclosure relates to methods and apparatuses for interacting with a patient. In particular, the present disclosure relates to a dialogue-based medical decision system to support a recovery, a (therapy) compliance and/or a coaching etc. of the patient.

### BACKGROUND

It is a key aspect of each therapy and/or rehabilitation that a patient closely pursues the recommendations of a doctor related to the therapy and/or the rehabilitation measures. Such measures, recommended to improve the health state of the patient are of particular importance after a long period of hospitalization of a patient and/or surgeries (e.g., by means of which medical devices are implanted) and/or injuries (e.g., traumata of the bones, etc.).

In a hospital and/or a rehabilitation clinic the required guidance and motivation for rehabilitation of the patient may at least in part be fulfilled by the employed medical staff. However, said measures are known as being rather labor intensive and thus also cost intensive which leads to an additional burden for (in particular) the public health system.

What is more, at least partly due to cost constraints, the patient often experiences a rather standardized therapy which is prescribed for a variety of patients in an almost identical manner. Such a therapy may mainly be based on the experience of a doctor but may not necessarily be optimized for the individual needs of the patients.

If a patient is discharged from a hospital, it is typically a critical aspect to still ensure that the patient fulfils the recommendations of the doctor (e.g., to abstain from certain foods) and to potentially conduct certain exercises (e.g., in the sense of a further rehabilitation). In particular, if the patient has returned to a known environment “old” and “common” habits (such as e.g., alcohol, unhealthy food, smoking, etc.) may be seen as a temptation to fall back into rather unhealthy habits. Said situation is further aggravated if the patient still suffers pain which may lead to certain avoidance actions (e.g., the avoidance of certain movements to avoid pain) which, however, do not lead to the desired treatment success. In extreme cases, this may lead to decompensations (e.g., of HF patients) not being recognized early enough.

There are systems that provide active support to comply with medication or training, for example. They may provide frontal feedback (now take your medicament XY) or training. However, a drawback of the currently known systems is that patients are not supported individually, customized to their needs.

Therefore, there is a need to improve accompanying a patient, e.g., during recovery, outside of the immediate range of influence of a doctor. In particular, there is a need to improve accompanying a patient at home during, e.g., the recovery process of the patient and to ensure patient compliance to a certain therapy. It is therefore desirable to identify optimal treatments, out of an ever increasing number of treatment options, and to minimize a treatment time and reduce medical costs.

The present disclosure is directed toward overcoming one or more of the above-mentioned problems, though not necessarily limited to embodiments that do.

### SUMMARY

According to an aspect of the present disclosure, the above need is at least partly met by a dialogue-based medical decision system which may comprise a receiver for receiving data associated with a health state of a patient sent from a device associated with the patient and a processing unit adapted to select a message associated with the health state of the patient based on the received data and based on stored patient data. Additionally, the system may comprise a transmitter for transmitting the message to the device and/or to another device associated with the patient. Thus, a doctor may be automatically assisted when coaching, verifying compliance and health state of a patient.

For example, the device associated with the patient may be a patient device. A patient device may be understood as a device which is associated with a patient, e.g., a device which is owned by the patient and/or carried by the patient and/or in the vicinity of the patient (e.g., a smartphone of the patient, a smart home device of the patient, a device associated with a medical device (implant) of the patient, etc.).

The device associated with the patient may, however, refer to any device which is capable of receiving and/or outputting data associated with the patient (e.g., health data). It may be a similar device as outlined above with reference to a patient device, e.g., a smartphone etc. of a doctor of the patient and/or of a relative of the patient.

For example, the data may be sent by a (patient) device, and the message may also be sent to the same (patient) device. The dialogue-based medical decision may thus track, based on the received data, a health state of the patient, and provide (the patient with) feedback based on the received data and based on already stored patient data. Hence, the present health state of the patient may be tracked. It may be taken into account when providing the message to the device. For example, based on data provided by a patient device, the patient device may be provided with a feedback message (e.g., an instruction, and/or a (follow up) question). For example, automatic dedicated coaching, and/or automated actively triggering compliance may be obtained.

Additionally of alternatively, the data may be sent by a device and then the message may be sent to another device. For example, the data may be sent by a patient device to determine the health state of the patient, and the message may be sent to the device of a doctor and/or a relative (e.g., indicating the current health state, a warning to take action, and/or an indication of whether or not the patient follows a therapy). The data may also be sent by another device (e.g., a device of a relative), e.g., to determine the health state, and then the message may be sent to the device of a doctor and/or the patient.

The dialogue-based medical decision system may be used to support a patient in a recovery process after e.g., a treatment in a hospital (e.g., after a surgery and/or a therapeutic treatment) which requires further guidance at home, where skilled medical staff may not always directly be available and/or present. The dialogue-based medical decision system may thus provide additional advice to accompany a patient during the recovery process by means of “talking” to the patient, based on input data provided by the patient and/or a doctor by means of the device (e.g., current blood values, blood pressure, pulse rate, a spoken sequence describing the health state of the patient, etc.) and stored patient data (e.g., medical/clinical records, therapeutic measures (a prescribed therapy and/or a medication, pre-existing diseases, data associated with an implant of the patient (e.g., current settings of the implant, recently acquired measures of vital parameters of the patient, etc.)). The medical dialogue-based decision system may be used to elaborate at least a part of a dialogue with the patient (as it will further be described below) and deciding, based on questions, provided to the patient, and the corresponding answers, of the patient, if the health state of the patient has improved or whether the health state of the patient has deteriorated.

In addition and/or alternatively, the dialogue-based medical decision system may also be understood as a coaching system for the patient. The dialogue-based medical decision system may be understood as a system to accompany a patient such that the patient follows a prescribed therapy, medication or the like. In such an application, the dialogue-based medical decision system may select the message such that the message comprises a question, to be provided to the patient, to ask the patient whether the patient has taken any prescribed drugs within a required time window and/or whether the patient has followed a prescribed (physical) exercise plan. The dialogue-based medical decision system may also be used to enquire the present health state of the patient by iteratively providing questions to the patient, wherein the provided question is selected based at least in part on a received answer, received from the patient.

In addition, it may also be possible that the dialogue-based medical decision system is used as a source of information for the patient. For example, if the patient is about to obtain an implant, the patient may have several questions regarding the associated surgery. In such a case, the data received from the patient may relate to a (medical) question. The dialogue-based medical decision system may then select a message, based at least in part on the received question, which may be seen as a response to the question of the patient (e.g., pertaining to the surgery and/or daily life with their condition). The answer (i.e., the selected message) may then be transmitted to the device by means of the transmitter. However, the dialogue-based medical decision system may not only be limited to questions regarding implants. The dialogue-based medical decision system may be used for any kind of health related questions of a patient.

Similar to that context, the dialogue-based medical decision system may also be used to support a (therapeutic) compliance of a patient. Therapeutic compliance addresses the aspect of ensuring that a patient pursues the requirements of a therapy, i.e., ensuring that a patient takes prescribed drugs, ensures enough physical exercises, ensures that a patient abstains from certain habits (e.g., alcohol consumption, smoking, etc.). In such a case, the data received from the device, may e.g., relate to data which is associated with the patient when performing a certain therapeutic exercise. The dialogue-based medical decision system may select a message to provide feedback to the patient based at least in part on the received data. Feedback may comprise a compliment for the patient that a certain exercise has been performed properly and/or an indication stat at least a part of the exercise has been performed wrong.

For example, a patient may be supported during HF (heart failure) therapy, e.g., in the cardiac rhythm management context, by the system. The system may actively ask about the patient's condition and forward the results of a corresponding dialogue to the attending doctor.

In another example, a patient may be supported during a neuronal pain therapy by the system. The system may actively ask about the patient's condition and forward results of a corresponding dialog to the treating doctor.

Moreover, the dialogue-based medical decision system may also be used to document the health state of the patient over time. For example, the documentation of the health state of the patient may be understood as a patient diary wherein, based at least in part on the received data associated with the health state of the patient (e.g., a comment of the patient), the dialogue-based medical decision system may store information associated with the patient for a certain day (or hour, etc.). The received data from the device may e.g., relate to health data which may be foreseen with a timestamp. The health data may comprise information that the patient suffers from high blood pressure on Monday whereas the dialogue-based medical decision system may document that the patient has fever on Wednesday. The received data may be stored in the dialogue-based medical decision system. A message may be selected and transmitted to the patient and/or a relative and/or a doctor of the patient in case a risky health state is indicated (e.g., defined by the experience of the doctor and/or ICD-10 guidelines). Since the received data may be foreseen with a timestamp, a documentation of the data may be allowed. A doctor of the patient (or generically any medical staff) may track the evolution of the health state of the patient over time and/or to adapt the message to the current health state.

In any case, the dialogue-based medical decision system may also be understood as a tutor for relatives of the patient. In such an application, the dialogue-based medical decision system may be used to train and/or tutor a relative of the patient such that a relative may be capable of accompanying the patient during a recovery process. In such a case the received data may be received from a device, carried by a relative of the patient and the received data may comprise a confirmation of a relative that the patient has performed a certain exercise and/or that the patient has followed a certain medication prescription. Based on the received data, the processing unit of the dialogue-based medical decision system may select a message associated with an indication that the prescribed medication was taken too late or too early. The dialogue-based medical decision system may transmit the selected message to the device of the relative to indicate to the relative how to improve the therapy compliance of the patient, e.g., by ensuring more carefully that a prescribed medication is taken according to an associated prescribed schedule.

The receiver may be understood as any means which is capable of receiving data associated with a health state of the patient, e.g., from a patient device. Said systems, associated with receiving data, may be implemented as RF electronics (e.g., an antenna for wireless reception of respective data, optionally including additional reception electronics (amplifiers, oscillators, etc.)). The receiver is preferably implemented to provide low-latency communication capability, e.g., by using, at least in part, WiFi, LTE, 5G, NFC, Bluetooth, etc. as it will further be described below. However, the reception of data may not only be limited to wireless reception but may also occur by means of a, preferably low latency, wired connection such as e.g., by ethernet, USB, serial, FireWire, HDMI etc. A receiver may not only be limited to a hardware implementation but may also relate to software implementations (e.g., logical implementations) such as e.g., one or more protocols (e.g., a socket) which facilitate the reception of data, e.g., from a patient device or other devices as outlined herein.

The transmitter may be understood as the counterpart of the receiver. Any implementation of the receiver, as outlined above, may also be applicable for the transmitter. Transmitter and receiver may be implemented as separate devices (configured respectively, i.e., for transmitting data) or may be the same device, e.g., a transceiver. By means of the above-mentioned implementation of the receiver, the dialogue-based medical decision system may transmit the selected message to, e.g., the (patient) device and/or to the another device.

In a preferred implementation of the receiver and the transmitter, the transmission and reception of data may be encrypted such that it provides data protection to the sensitive health related data to be communicated between, e.g., the device and the dialogue-based medical decision system.

The processing unit may be understood, in an exemplary and simple implementation, as implementing a decision tree. The decision tree may possess at least one layer (hereinafter the number of layers will be referred to as the depth of the tree). The decision tree may further comprise at least one particular question to be transmitted to the device. The at least one question (i.e., the selected message as described above) may be understood as the leaf of the decision tree at a certain depth of the tree. The decision tree may be adapted such that it possesses at least one branching originating from at least one of the leaves. The branching may be understood as potential disjunct answers received from a patient in response to a question transmitted to the patient (in the form of a selected message). In other words, with each asked question by the dialogue-based medical decision system and with each received answer, the subsequent questions (to be transmitted to the patient) may be found on the subsequent depth of the decision tree. Therefore, a pre-defined dialogue may be pre-implemented in the processing unit, wherein each of the leaves corresponds to a certain sequence of a dialogue. By parsing through the decision tree, a dialogue with the patient may be established. Each of selected messages may thus also consider any previous response of the patient and any message transmitted to the patient beforehand. It may further be possible that more than one decision tree (each associated with a dialogue) is stored in the dialogue-based medical decision system and/or a remote server, wherein each of the decision trees is associated with a context-specific dialogue. In other words, there may be a decision tree for ensuring a regular medication of the patient and there may be a separate decision tree for surveilling the current health state of the patient. However, it may also be possible that said aspects are merged into a single decision tree. The at least one decision tree may be pre-programmed and/or may be pre-implemented by a doctor (e.g., by means of a dedicated user interface, e.g., a graphical user interface, which may be accessible by means of an internet browser and/or an app etc.) and/or with the help of an artificial intelligence.

The dialogue-based medical decision system may be a server-based system.

The device may e.g., relate to a smartphone, wearable (e.g., a smart watch and/or a chain with a wearable sensor system), a device in communication with a medical device (implant), or any other suitable electronic device which may be associated with (the health state of) the patient. Alternatively, a (patient) device may also relate to a stationary device which is capable of being associated with the health state of the patient. In such a case, the device may e.g., be a smart home device (e.g., a smart speaker, a smart TV, etc.). Even though certain features, described below, are referred to the patient device, they are nevertheless equally applicable to any device associated with the health state of the patient, i.e., a device carried by a doctor and/or a relative etc.

The another device may be configured similarly as the (patient) device (as outlined above) and may be associated with a relative and/or a doctor of the patient. In particular, the device (from which data associated with the health state of the patient is received) and the another device may be different devices. It may thus be possible that the device is carried by the patient whereas the selected message is transmitted to a relative and/or a doctor of the patient. In such an application, a relative and/or a doctor may be warned that a current health state of the patient is unhealthy or a confirmation may be sent to a relative and/or a doctor that the recovery process of the patient is satisfying. Such device may also be used to provide data (associated with the health state of the patient) to the dialogue-based medical decision system and/or may output data (associated with the health state of the patient) as received from the dialogue-based medical decision system. Moreover, another device may provide additional data associated with the health state of the patient to the dialogue-based medical decision system, e.g., from the subjective/objective perspective of a relative (e.g., living with the patient) and/or a doctor. Additionally or alternatively, such another device may also be used as a target for any transmission of the dialogue-based medical decision system to a relative and/or a doctor of the patient. Based thereon, the another device may also be understood to be associated with the health state of the patient.

The device and/or the another device may additionally or alternatively be adapted to communicate with an implant and/or a subcutaneous device. In such a case, a device may refer to a smartphone, a wearable and/or a smart home device. In general, a (patient) device may also relate to any IoT device which is capable of receiving a patient input (e.g., a spoken sequence) and which is capable of outputting a received message from the dialogue-based medical decision system to the patient (e.g., by means of a display, a LED, a loudspeaker, etc.).

The device may thus be capable of receiving data from an implanted pacemaker and/or any other type of implant. The device may e.g., be a smartphone (as outlined above), equipped with e.g., an app to receive and forward (i.e., transmit) the implant data to the dialogue-based medical decision system. The communication between the device, the implant and/or the dialogue-based medical decision system may in any case e.g., be implemented as a Bluetooth connection and/or an NFC connection.

The device may further be equipped with an interface for obtaining an input of a patient and/or a relative and/or a doctor. Such an interface may e.g., relate to at least one microphone which may be used to record a spoken sequence of the patient and/or a relative and/or a doctor and/or generically any sound associated with the health state of the patient (e.g., a breathing noise, a cough noise, etc.). For example, the device may comprise a microphone for such input (and/or a graphical user interface, such as a touch screen and/or a screen and keys, etc.). In addition, the device may also comprise at least one sensor which is capable of measuring at least one parameter associated with the health state of the patient (e.g., a pulse rate, a body temperature, etc.). The data sent to the decision system may be based on and/or include such input and/or parameter.

A message, which may be selected by the processing unit, may relate to a message which is associated with the health state of patient. Such a message may e.g., be a pre-stored question (e.g., “How do you feel today?”) and/or a phrase, e.g., a statement or instruction (e.g., “Please take one pill of ibuprofen.”). The selected message may also relate to a message which indicates to a relative and/or a doctor the current health state and/or whether the patient is in a satisfying recovery state or whether a deterioration has occurred. The target receiver of the message may thus be the patient and/or a relative and/or a doctor of the patient. The pre-stored question and/or phrase may be related to the health state of the patient (e.g., the current subjective feeling (question) and/or a statement that the patient should take a certain medication immediately in order to remain in agreement with the prescribed therapy). The pre-stored question and/or phrase may be selected from a set of pre-stored questions and/or phrases for different application scenarios. For example, at least one question and/or phrase may be directed to the subjective feeling of the patient while at least one other stored question and/or phrase may relate to the aspect whether the patient has taken the prescribed medication. Moreover, it may be possible that the at least one stored question and/or phrase relates to a recent surgery of the patient and/or a specific type of implant of the patient. In the latter case, the dialogue-based medical decision system may ask questions to characterize whether the implant is configured such that the patient has a subjective and/or objective healthy feeling.

The at least one message may be stored in a text format in the dialogue-based medical decision system and/or at a remote place as will further be described below. Additionally, the at least one message may also relate to a stored audio and/or video output, e.g., a pre-stored spoken question (and/or a video sequence) which may be transmitted to the (patient) device from the dialogue-based medical decision system. The at least one message may also relate to at least one stored command which is adapted to cause an LED (or any other suitable indication) on the (patient) device to flash/blink. This may be understood by the patient to take a certain action (e.g., take a certain drug).

The selection of the message may be based on received data from the patient and/or a relative and/or a doctor. The received data may allow to characterize the current health state of the patient (e.g., by considering at least one parameter associated with the health state). Based thereon, the selected message may be selected context-specific, i.e., such that the selected message is in conjunction with the current health state and/or a therapy of the patient. The selection may further comprise selecting the message based on stored patient data. For example, if the received data indicates that the patient suffers from nausea, based on the stored patient data, it may be concluded that this is a side effect of the surgery that can be remedied by taking a certain action. The system may then transmit a message to the (patient) device including an instruction to take that action. In other examples, the patient data may indicate, based on the stored patient data, an emergency situation, and a corresponding message may be transmitted to the doctor.

As a further example, the selection of the message may e.g., comprise a determination of a current health state based on the received data and the stored patient data. For example, a patient input, e.g., the spoken sequence of the patient “I feel very exhausted today” may be received, and additionally the current location of the patient, the current weather situation and the pre-existing diseases of the patient may be considered. In such a case, the dialogue-based medical decision system may first determine that there is considerable risk that the patient suffers from a heart failure (due to e.g., a pre-existing disease), and an emergency message may be sent to relatives, the doctor (e.g., in an emergency unit). In other instances, the dialogue-based medical decision system may determine that the present fatigue of the patient may be a result of the environmental conditions (e.g., the location in a high humidity climate zone). In response to this determination, the system may select a message which states that the patient should take a certain drug to overcome the present fatigue symptom.

The selection of the message may also refer to indicating whether the patient maintains the prescribed therapeutic compliance (and/or to which extent the patient maintains the therapeutic compliance). The message may e.g., be sent to a relative and/or a doctor of the patient as a surveillance measure directed to a satisfying recovery process. The message may potentially also indicate whether the patient is in an emergency situation. If it turns out that the patient is in an emergency situation (e.g., if it is determined that the patient may be at risk to suffer an heart attack), the dialogue-based medical decision system may forward a threat warning to the (patient) device and/or the another device.

The stored patient data may be stored in a database and/or a blockchain. The stored patient data may be stored on the dialogue-based medical decision system (e.g., a non-volatile storage medium thereof) and/or a remote server. In any cases, the stored patient data may be encrypted to ensure the data security of the health state related data.

Stored patient data may exemplarily comprise general patient data (birthdate, gender, etc., the current location of the patient, weather information, etc.). It may also comprise health data, such as pre-existing diseases, any kind of medical/clinical records, therapeutic measures (medical indications, a prescribed therapy and/or a medication, HF therapy, pacemaker implantation etc.). It may also be possible that the stored patient data comprises data associated with an implant of the patient (e.g., current settings of the implant, recently acquired measures of vital parameters of the patient, etc.). This data may be entered for a specific patient by an operator of the system that initializes support for a patient. Additionally or alternatively, the corresponding data may also be automatically retrieved, e.g., via an electronic health monitoring system or from electronic health records or patient files. Also an operator may select, in the system, one or more specific purposes for a specific patient, e.g., purposes as outlined herein (e.g., introduction into a therapy (e.g., CRM therapy, neurologic pain therapy, etc.), general information on daily life with a specific diagnosis and/or therapy, event-based support of compliance (e.g., reminder concerning medication intake, recording a patient diary, determining a decompensation and informing a doctor, determining a change of behavior and informing a doctor).

The transmitter of the dialogue-based medical decision system may further be adapted to transmit information, associated with the health state of the patient, to the (patient) device such that the (patient) device is enabled to base the sent data on the information. It may be possible that the device transmits data associated with the health state of the patient in response to received information, transmitted from the dialogue-based medical decision system to the device. In such a scenario, the dialogue-based medical decision system may initiate a dialogue between the device and the dialogue-based medical decision system. For example, the patient and/or relative may be asked individual questions, e.g., based on the stored patient data, to determine the current health state of the patient. The corresponding data sent from the device may then be used to determine the current health state, and further follow-up questions may be provided to either determined the health state in further detail, and/or to tailor the message (e.g., assisting the patient in therapy, medication etc.) to the current health state.

The dialogue-based medical decision system may further be adapted to receive the data in the form of audio and/or visual data and/or to transmit the message comprising audio and/or visual data. It may be possible that, if the selected message is stored in a text format, that the stored message is converted into a spoken sequence by a message-to-audio-converter. Similarly, received (voice) data may be converted into text by such converter.

Such a converter may e.g., be part of the dialogue-based medical decision system or may be implemented as a cloud service running on a remote server system, e.g., in communication with the dialogue-based system. It may be possible that the processing unit forwards the selected message to a respective cloud service for message-to-audio conversion. The processing unit may then receive/obtain audio data (e.g., a file) from the message-to-audio-converter which may then be transmitted to the (patient) device and/or the another device. It may also be possible that the message-to-audio-converter is part of the processing unit such that the audio file may directly be transmitted to the (patient) device. When implementing the message-to-audio-converter in a cloud, the computation power (necessary for the conversion of the message to an audio file) at the (patient) device may be minimized, thus reducing battery power consumption and hardware resources. In any case, it may be seen as an advantage that the messages are stored in text format to minimize the associated storage space.

Moreover, the dialogue-based medical decision system may also be adapted to receive audio data (e.g., an audio file) from the (patient) device and/or any other device associated with the patient (or also from the another device). In such a case, an audio-to-message converter may be foreseen which converts the received audio file into a processable text file which may be used to select a subsequent message (to be transmitted to the patient) by means of the processing unit. The reception of an audio file may be understood as the inverted process of transmitting a certain question to the patient. Further implementation details for the audio-to-message converter may be identical to the implementation of the message-to-audio-converter. It may be possible that both converters are implemented in the processing unit and/or a remote cloud or that only one of the two converters is implemented in the processing unit whereas the respective second converter is implemented in a remote cloud. If the received data and the transmitted message are both implemented as audio data or audio files, the communication between the dialogue-based medical decision system and the (patient) device (and/or the other device which is associated with the patient and/or the another device), may be understood in analogy to a telephone call between the dialogue-based medical decision system and the (patient) device. The telephone call may preferably be encrypted. The cloud-service for the message-to-audio conversion and/or the audio-to-message converter and the processing unit of the dialogue-based medical decision system may be in communication over the internet (wireless or wired), a local area network (LAN), a wide area network (WAN), etc. It may also be possible that the patient data relates to a wearable or an IoT device, etc. (as outlined above) indirectly received by the system via the (patient) device, and/or (additionally) received directly from a wearable, an IoT device, etc.

The receiver of the dialogue-based medical decision system may further be adapted to receive audio input data and/or visual input data associated with the patient. It may also be possible that the device and/or any other device associated with the patient may comprise a camera. Such a camera may be adapted to capture an image of the patient, e.g., an image of at least a portion of the skin of the patient if the patient responds to the device that she/he feels itch, potentially accompanied by a rash. That combination may be understood as a potential indication for an allergic reaction of the patient, e.g., caused by a certain medication and/or life circumstances (e.g., an allergic reaction caused by pollen). As a consequence, if the image of the rash is received by the dialogue-based medical decision system, the processing unit may inter alia identify the rash as a rash potentially caused by an allergic reaction. In both cases, it may be possible that a microphone and/or the camera is part of the (patient) device and/or may be implemented as a separate device which is in communication with the (patient) device. By incorporating further stored patient data, the processing unit may potentially be aware of a known allergy of the patient, e.g., against pollen. As a result, the message may be selected such that the dialogue-based medical decision system recommends the patient to take a certain drug (e.g., an anti-histamine) and/or the message may be selected such that it can be excluded that the patient is at risk (e.g., due to an anaphylactic shock).

In addition or alternatively, it may also be possible that the device does not only capture a single image but may also capture a video sequence of the patient. In such a case, the dialogue-based medical decision system may be adapted to receive a video sequence as visual input. Such a video sequence may be processed similarly to a single image capture (as outlined above) and may e.g., allow the determination if the motion sequence of the patient during walking is satisfying or whether the patient shows any kind of indication of abnormality, e.g., that may indicate a malfunctioning of an implant, etc.

In one embodiment, the system may be further adapted to receive audio data and/or visual input data associated with the patient, for example, an image and/or a video sequence of the patient and/or patients skin portion, e.g., captured by a camera. The advantages mentioned above for partial aspects of this embodiment also apply to all parts of this embodiment.

The video sequence may additionally or alternatively be used as a surveillance monitor whether the patient performs prescribed physiotherapeutic exercises (which may be executed at home) properly or whether the patient follows a potential erroneous motion sequence. In such a case, the video sequence may also be received by the dialogue-based medical decision system. Based on an evaluation of the video sequence by the processing unit, it may be determined that the patient follows indeed an erroneous motion sequence which may potentially cause additional injuries. In such a scenario, the message (which is transmitted to the (patient) device by means of the transmitter) may be selected such that the patient (and/or a relative and/or a doctor) is informed that the current motion sequence is wrong. The patient may thus also receive at least one indication/recommendation what to change in order to be in accordance with the prescribed physiotherapeutic treatment. It may also be possible that the message includes a video sequence which is transmitted to the (patient) device (from the dialogue-based medical decision system) and which e.g., shows how to perform the prescribed exercises properly. It may also be possible that the video sequence is transmitted to the device of a relative of the patient such that the relative and the patient may collectively ensure that the exercise is performed correctly. In such a case, the (patient) device (and/or another device associated with the patient) may possess a built-in screen to display the video sequence transmitted to the respective device and/or may possess means to output the video sequence to a screen (e.g., a smart home device which is equipped with an HDMI port to be connected to a TV screen). In any case, audio and/or video data may relate to the respective audio and/or video file and/or alternatively to a bit-stream of the respective file, i.e., a serialization of the respective file.

The dialogue-based medical decision system may be adapted such that the processing unit comprises an artificial intelligence. As an alternative or accompanying the decision tree (as outlined above), the message may be selected based on received data (associated with the health state of the patient) and/or on stored patient data by means of an artificial intelligence. The selection of the message may comprise the consideration of at least one pre-asked question (i.e., a pre-transmitted message) to the patient and/or the received data from the patient. In such a scenario, the selection of the message may comprise the “history.” of the dialogue. Alternatively, it may also be possible that the message is only selected based on the received data from the (patient) device, wherein the “history” of the dialogue is essentially neglected.

The processing unit of the dialogue-based medical decision system may continuously and iteratively be improved by (further) training the underlying artificial intelligence with pairs/matches of questions and answers (e.g., as recorded from real patient/doctor interactions and/or associated patient data from a health file). The training data are preprocessed, such as to ensure anonymous training. The goal of such a procedure may be seen in allowing a more sensitive selection of the message to be provided to the patient in a subsequent dialogue step/sequence. To achieve a continuous training of the artificial intelligence, the processing unit of the dialogue-based medical decision system may comprise a natural language processing unit which may be capable of recognizing the sense of a spoken sequence, i.e., the natural language processing unit may be adapted to recognize whether the patient suffers of pain based on the wording of the spoken sequence. The artificial intelligence may be implemented as a neuronal network, e.g., at least one of a feed-forward-network or a recurrent network. A neural network is a software algorithm which is able to generate a model based on training data to provide predictions or decisions (without expressly being programmed for that). For example, Deep-Learning may be used to train the network. Each of said networks may comprise at least one hidden layer which may be associated with a parameter of the patient (e.g., a blood pressure, pulse rate, etc.). The artificial intelligence may then be used to assign an input value (e.g., a spoken sequence of the patient) to an output value of the artificial network (e.g., a selected message). The hidden layers may be foreseen with weighting factors. If several hidden layers are used e.g., in sequence, each of the hidden layers may be weighted by means of a respective weighting factor. The output of the neuronal network may then be understood as a weighted result of the individual hidden layers. Each of the hidden layers may contribute to the output value to a different extent based at least in part by means of the chosen weighting factor. Said procedure may allow to define the importance of an individual hidden layer (and the associated parameter which is represented by the hidden layer) and the desired effect on the calculated output. The individual hidden layers may either be predefined, e.g., by a programmer of the respective neuronal network and/or the patient and/or a doctor and/or a relative of the patient. In case, recurrent neuronal networks are used, it may be possible to backreference hidden layers. In other words, during processing of an input of a recurrent neuronal network, it may be possible that it is decided at the n-th processing step, that the processing should once again reconsider the n−2th processing step. Therefore, a recurrent neuronal network may be understood as neuronal network comprising loops.

Additionally or alternatively, it may also be possible to train the neuronal network. Training may be understood as a procedure during which several pairs of input and output values may be presented to the neuronal network. Training algorithms may be used to then define the at least one weighting factor associated with the at least one hidden layer which is required to transform the presented input into the presented output value of the neuronal network. When training a neuronal network in the context of the present disclosure, the network may be trained with at least one of general patient data (e.g., age, gender, medical indications, weight, etc.), pre-existing diseases of the patient, therapeutic measures (e.g., administered physiotherapeutic treatments), a medication plan (e.g., a schedule which drugs are required on a certain day of the week and the respective time they have to be taken), localization information (e.g., GPS data of the patient and/or the (current) assignment of a mobile device to a respective base station (wherein the localization information of the base station is known)), motion profiles of the patient (e.g., the average distance a patient walks per day, optionally including associated localization information), weather data and calendar dates (e.g., public holidays, religious holidays, birthdays, etc.), level of education of the patient (e.g., the highest academic degree of the patient), potential therapeutic measures for the patient (e.g., a variety of physiotherapeutic exercises to be carried out by the patient, information on implants (e.g., pacemaker implants)), etc. The training data may be presented to the neuronal network at once (prior to using the dialogue-based medical decision system in communication with the patient) or may be presented to neuronal network either step-wise by the patient and/or a relative and/or a doctor. In the latter implementation, a step-wise learning will be obtained. Optionally, the neural network comprises a model control layer, such as to make it easier to verify and monitor the model.

In addition or alternatively, and in particular if images and/or video sequences are to be evaluated (e.g., to detect a motion sequence of the patient during a physiotherapeutic exercise) the neuronal network may also relate to a multidimensional network, e.g., a convolutional neuronal with three dimensions, for example. In such a case, the convolutional neuronal network may comprise at least one convolutional layer (the number of convolutional layers may also be referred to as the dimension of the convolutional network). A convolutional layer may be understood as means for recognizing certain patterns (e.g., the position of an arm and/or foot of the patient) in the raw data supplied to the convolutional neuronal network. Subsequent to the at least one convolutional layer, at least one pooling layer (i.e., a subsampling layer) may be applied to the recognized patterns. This allows to reject unnecessary recognized information and may decrease the overall amount of data without affecting the performance of the convolutional neuronal network. When processing audio, image and/or video data, the convolutional neuronal network may at least be a two-dimensional network.

The artificial intelligence may be trained with processed data (anonymous training), and the training data requirements will depend on the desired function of the system. Training data may, for example, be pairs/matches of questions and answers (e.g., as recorded from real patient/doctor interactions and/or associated patient data from a health file), apart from the data already outlined above.

The artificial intelligence may be part of the processing unit. The artificial intelligence may be implemented within the dialogue-based medical decision system and/or may be implemented externally. If implemented externally, the artificial intelligence may be executed in a cloud-based system on at least one server. A cloud-based system may provide the advantage of a load distribution (if the dialogue-based medical system experiences high traffic), may be more robust against cyber-attacks (e.g., DDOS attacks), decreases latency (i.e., the response time between a patient request (a question by the patient) and the selection of a corresponding message), may allow the access to larger amounts of data (e.g., with respect to patient data and/or stored messages) and generically increased computation power. To put it in a nutshell, a neuronal network may be used to decide, based at least in part on questions (which have already been provided to the patient) and/or input from the patient (e.g., respective answers), the clinical record of the patient, etc. which message or question should be provided next to the patient to best and quickly obtain the desired information regarding the current health state of the patient and/or to provide the patient with the required feedback.

The processing unit of the dialogue-based medical decision system may further be adapted to select the message from a set of stored dialogues. As mentioned above, the processing unit may foresee at least one stored dialogue. A stored dialogue may refer to at least one stored sequence (e.g., a question of the dialogue-based medical decision system to the patient: “How do you feel today?”) of the potential dialogue between the dialog-based medical decision system and the patient and a potential respective answer of the patient and a potential reaction of the processing unit (i.e., a next questions and/or the transmission of an indication that a deterioration of a health state of the patient occurred to a relative and/or a doctor of the patient). Preferably, the described sequence is performed at least twice. The at least one stored dialogue may relate to a respective decision tree as mentioned above and/or may be selected by the artificial intelligence as described above. The dialogue-based medical decision system may receive data from the patient (by means of the (patient) device), e.g., an answer, which may at least in part be used as a basis to determine a potential (next) question for the patient from the set of stored dialogues. Said dialogue may be performed until a satisfying set of information, characterizing the health state of the patient, has been determined. A satisfying set of information may e.g., be pre-defined by e.g., a doctor such that a reliable conclusion with respect to the current health state and/or of the therapy compliance of the patient may be facilitated. The satisfying set of information may e.g., be based on the experience of a doctor. As mentioned above, the set of stored dialogues may e.g., be stored on the dialogue-based medical decision system and/or may be stored remotely, e.g., in a cloud. In any case, the stored dialogues may be stored in text format and/or may be stored as audio and/or video files. Selecting a message from a set of stored dialogues may facilitate a patient-specific dialogue concerning the health state of the patient by iteratively asking questions to achieve a satisfying characterization of the health state of the patient in a potential question-and-answer procedure. The dialogue may be patient-specific, as the dialogue according to the present disclosure, is not pre-defined but may individually be developed according to the current needs of the patient, i.e., the at least one question provided to the patient, may be different when the question-and-answer procedure is performed repeatedly (e.g., once a day) as the answers and the health state of the patient may change, just as the stored patient data. The at least one question may also be different if the system interacts with different patients. The set of stored dialogues may be stored on the dialogue-based medical decision system and/or on a remote server and may be accessible over the internet and/or a LAN, WiFi, 5G, etc. The set of stored dialogues may in any case be stored in a database and/or a blockchain to ensure a fast, redundant and secure data recovery.

The dialogue-based medical decision system may be configured to transmit the message to a message-to-audio-converter prior to transmitting the message to the device and/or the another device.

Additionally and/or alternatively, the (patient) device may also possess a message-to-audio-converter. In such a case, the message, received by the (patient) device, may be converted into audio on, e.g., the (patient) device. There may be default settings at the (patient) device indicating how to convert the message to audio (e.g., language, gender of the voice, etc.).

The dialogue-based medical decision system may further be adapted to transmit information to enable an output of the message by the device and/or by the another device as an audio output and/or as a visual output. If the dialogue-based medical decision system is in communication with a cloud service that may allow to convert a stored message (e.g., in text format) into an audio file as outlined above, the information may comprise language settings for the selection of the message (in case the dialogue-based medical decision system is implemented as a multi-lingual device), the gender of the voice associated with a spoken audio file, the academic degree of the patient (e.g., to allow a target group adequate phrasing of the dialogue), etc. Said information may e.g., be considered when converting the selected message into an audio file. It may e.g., be possible that the message-to-audio converter also comprise translation capability for multi-national usage of the dialogue-based medical decision system. It may be understood that the information is then transmitted from the dialogue-based medical decision system to the respective cloud service for the message-to-audio conversion. The dialogue-based medical decision system may thus provide means for a respective transmission unit which pre-converts the audio data (e.g., applying a certain audio codec) to be transmitted such that it fulfils the standard requirements for the desired transmission method (e.g., WiFi, 5G, Ethernet, etc.). Moreover, the audio data may be encrypted prior to its transmission, may be serialized (i.e., converted into a bitstream) and may then be converted into the respective transmission format by means of the relevant standard and protocols (TCP, UDP, etc.). The same may account for visual outputs which may generically refer to the transmission of image files and/or video files. It may also be possible that the information is transmitted along with the selected message such that the information may be understood as a configuration file by the device. In such an application scenario, the device may perform the message-to-audio conversion. In such a case the (patient) device may convert the received message from the dialogue-based medical decision system into an audio file, based at least in part on the information, which may then be output to the patient (or relative or doctor, respectively).

The dialogue-based medical decision system may further be implemented in a cloud. It may thus be possible that not only the artificial intelligence and the message-to-audio-converter is implemented in a cloud but that the entire dialogue-based medical decision system is implemented in a cloud, e.g., as a Software-as-a-Service (SaaS) and/or Hardware as a Service (Haas) solution. Such a system may provide a variety of advantages as outlined above. The implementation of the dialogue-based medical decision system may in particular provide the advantage of a client-server-architecture wherein the (patient) device (and/or another device associated with the patient) represents the client while the dialogue-based medical decision system may be seen as server unit(s). The client device may thus be understood as a data input/output system which may provide e.g., a spoken audio sequence to the cloud system (i.e., the dialogue-based medical decision system) for further processing. The processing may lead to the selection of a message based at least in part on the received data from the (patient) device and stored data of the patient as outlined above. The cloud system may be operated in a certain hospital environment, a dedicated university research network or may be part of the internet. It may also be possible that the cloud system is implemented on the (patient) device as a service, such that it may be capable of receiving data from the patient, e.g., by means of a certain port. It may then also be possible to transmit from the cloud service (i.e., the dialogue-based medical decision system) to the (patient) device. Such an implementation may decrease the required computation power for the (patient) device and the energy consumption of the (patient) device.

The receiver and/or the transmitter of the dialogue-based medical decision system may further be adapted to communicate based on a low-latency communication system. A low-latency communication system may be understood as a communication system which is able to provide a fast answer on the downstream if a query is sent to a server on the upstream. Fast in this context may be understood as relating to any communication during which an operator (e.g., the patient) does not recognize any latency. In other words, a low-latency communication system may relate to communication systems for which an operator does not perceive any waiting time in between the transmission of a message and the reception of a respective response, similarly as in a phone call. For example, a latency may be less than 0.5 s, less than 200 ms, less than 100 ms or even less than 50 ms. Such low-latency communication systems may exemplarily relate to WiFi, 5G, LTE, mmW, Ethernet, fibre communications, etc. Low-latency may not only to be understood as low-latency on the physical layer but also on the protocol and hardware layer.

Based on the low-latency, the decision system may provide decisions in real-time, e.g., select reply messages and/or messages containing follow-up questions and/or alerting/informing relatives and doctors in real-time.

The dialogue-based medical decision system may further be adapted to be activated based at least in part on a request from the device and/or the another device and/or a pre-defined schedule. The dialogue-based medical decision system may be activated based at least in part by the patient. In such a scenario, the patient may ask the dialogue-based medical decision system a question, i.e., “I feel vertigo today, what can I do?”. Said question may be received by the dialogue-based medical decision system as outlined above. A message may be selected by the processing unit of the dialogue-based medical decision system, e.g., including a follow-up question to find out more about the patient's state. In such a case, the articulated question of the patient may be understood as a trigger for starting a dialogue in between the patient and the dialogue-based medical decision system. The dialogue-based medical decision system may then be understood as a coach for the patient, capable of answering the health state related questions of the patient. For example, the patient may ask the dialogue-based medical decision system whether two drugs (medications) may be taken simultaneously without risking any unwanted side effects. In addition or alternatively, it may also be possible that the patient activates the dialogue-based medical decision system by pressing a button, e.g., in soft- and/or hardware on a smartphone. The dialogue-based medical decision system may then start to select and transmit an opening sequence for a dialogue to the (patient) device, e.g., “How do you feel today?”. In response to the answer of the question, the dialogue-based medical decision system may iteratively select subsequent messages to characterize the current health state of the patient as outlined above.

In addition or alternatively, it may also be possible that the dialogue-based medical decision system is activated by a relative (of the patient). In such a case a relative may e.g., activate the dialogue-based medical decision system by means of e.g., an app on the device. A motivation for the activation of the dialogue-based medical decision system by a relative may e.g., be to FIGURE out whether the aged parents and/or grandparents are at a satisfying health state and whether the aged parents and/or grandparents still pursue a prescribed therapy. In response to e.g., such a remote activation, the patient device (which is carried by the patient and/or at least close to the patient) may be supplied with respective questions and/or dialogues to characterize the health state of the patient. The results obtained from the patient may also be transmitted to the relatives and/or to a doctor.

In addition or alternatively, it may also be possible that the dialogue-based medical decision system is activated by a doctor (of the patient). A doctor may pursue a similar goal with respect to the health state of the patient as her/his relatives. A doctor may activate the dialogue-based medical decision system as a routine checkup for the patient, e.g., on a regular basis (as it will further be outlined below) and or ad-hoc (e.g., only once).

In addition or alternatively, it may also be possible that the dialogue-based medical decision system is activated based at least in part on a pre-defined schedule. A pre-defined schedule may e.g., relate to a treatment plan of the patient. The treatment plan may comprise one or more check-ups over the range of one year. The dialogue-based medical decision system may then be activated based at least in part on said pre-defined dates.

The dialogue-based medical decision system may automatically be activated periodically. Periodically in the context of the present disclosure may be understood as occurring based on a recurring time interval such as e.g., at least one of once per hour, once per day, once per week, once per month, once per year, etc. In such a case a dialogue may be initiated with the patient, directed to the health state of the patient, based at least in part on the pre-defined periodicity of the activation.

In addition or alternatively, the dialogue-based medical decision system may be (automatically) activated based on a detected event, e.g., based on sensor data of one or more sensors associated with the patient. For example, it may be possible that the dialogue-based medical decision system is activated based on received sensor data, wherein the sensor data is associated with the health state of the patient. For example, a patient may carry at least one sensor, e.g., in the patient device or in communication with the patient device, such as e.g., in a smartwatch, or in an implant (e.g., a pacemaker). Said smartwatch may measure the pulse rate of the patient e.g. once per minute. If the smartwatch detects an abnormal pulse rate (e.g., >180 bpm) of the patient, the smartwatch may indicate the abnormality to the dialogue-based medical decision system. The dialogue-based medical decision system may then initiate a question-and-answer procedure as outlined above to characterize the current health state of the patient. Additionally or alternatively it may also be possible that the dialogue-based medical decision system periodically receives sensor data. Additionally or alternatively, it may also be possible that the dialogue-based medical decision system receives sensor data in a context driven manner. For example, the dialogue-based medical decision system may only receive sensor data if the ambient temperature around the patient is above 30° C. In such a scenario, the dialogue-based medical decision system may receive sensor data from e.g., an implant of the patient (e.g., from a pacemaker) indicating the current health state of the heart of the patient.

Other aspects of the present disclosure may relate to a device associated with a patient. The device may comprise a transmitter configured to transmit data associated with a health state of the patient to a dialogue-based medical decision system and a receiver configured to receive, from the dialogue-based medical decision system, a message associated with the health state of the patient based on the transmitted data and based on patient data stored by the dialogue-based medical decision system. Moreover, the device may comprise an interface adapted to indicate the received message to the patient. The transmitter and the receiver of the device may be understood in analogy to the transmitter and the receiver of the dialogue-based medical decision system as outlined above. The processing unit of the device may relate to a processing unit which processes the received data from the dialogue-based medical decision system such that the device is capable of indicating and/or displaying the received data. For example, the processing unit of the device may be adapted to decode the received data stream such that the device is capable of displaying the received video stream (e.g., showing the patient certain physiotherapeutic exercises to be performed by the patient) and or to cause a LED of the device to start flashing which may be understood as an indication for the patient and/or a relative and/or a doctor to take certain drugs. Such a device may also be used to (remotely) track and characterize the health state of the patient.

Aspects of the present disclosure further relate to a method implemented by a dialogue-based medical decision system. The method may comprise receiving data associated with a health state of a patient from a device associated with the patient and processing the data to select a message associated with the health state of the patient, based on the received data and based on stored patient data. Moreover, the method may comprise transmitting the message to the device and/or another device associated with the patient and/or a relative and/or a doctor of the patient.

The method steps may be adapted to be executed at least two times to converse with the patient and/or a relative of a patient and/or a doctor of a patient. As outlined above, it may be a preferred implementation of the present disclosure to converse with the patient in a dialogue. When executing the method steps (as outlined above) repeatedly (i.e., at least twice), a dialogue between the patient and the dialogue-based medical decision system may iteratively be achieved. This may provide the advantage of a more detailed characterization of the current health state of the patient as more health state related parameters may be obtained due to the conversion with the patient, and the system may ask questions tailored to the present situation to determine the health state in a refined manner.

In some examples, the patient may be provided, by the system, with a question, and the data sent by the patient device may be based on the question. The system may then possibly follow-up with one or more further questions (message) and receive corresponding further data from the patient device and/or another device. Based on the received data, the system may provide a message to the another device (e.g., with information on the health state, such as well-being, compliance with therapy etc.) and/or a message to the patient device (e.g., feedback to comply with therapy, etc.).

Aspects of the present disclosure further relate to a method implemented by a device associated with a patient. The method comprises transmitting data associated with a health state of the patient to a dialogue-based medical decision system and receiving, from the dialogue-based medical decision system, a message associated with the health state of the patient based on the transmitted data and based on patient data stored by the dialogue-based medical decision system. Moreover, the method at the device may comprise indicating the received message to the patient and/or a relative and/or a doctor of the patient.

The transmission of the data associated with the health state of the patient may be associated with a spoken sequence of the patient and may be converted into a text message by means of a respective audio-to-message-converter. Such an audio-to-message-converter may be part of the device and/or may be provided to the device by means of a remote server. The device may be in communication with the server, preferably by means of low-latency communication systems as outlined above. It may also be possible that the device transmits the spoken sequence of the patient as audio data to the dialogue-based medical decision system. It may also be possible that the data associated with the health state of the patient relates to a text message, input by the patient, and/or to image and/or video data. A similar procedure, as outlined above, may relate to the indication of the received message. The received message may be received as audio data and/or image data and/or video data and/or a text message and/or a command to perform a certain action (e.g., cause a LED of the patient device start flashing). The received message may either directly be output to the patient and/or may be converted from text to audio (as outlined above).

The present disclosure further relates to a computer program, comprising instructions which, when executed, cause a computer to perform the steps of any of the methods as outlined herein.

Also, the present disclosure further relates to a non-transitory computer-readable medium comprising a computer program, comprising instructions which, when executed, cause a computer or processor to perform the steps of any of the methods as outlined herein.

Whether described as method steps, computer program and/or means, the functions described herein may be implemented in hardware, software, firmware, and/or combinations thereof. If implemented in software/firmware, the functions may be stored on or transmitted as one or more instructions or code on a computer-readable medium. Computer-readable media includes both computer storage media and communication media including any medium that facilitates transfer of a computer program from one place to another. A storage medium may be any available media that can be accessed by a general purpose or special purpose computer. By way of example, and not limitation, such computer-readable storage media can comprise RAM, ROM, EEPROM, FPGA, CD/DVD or other optical disk storage, magnetic disk storage or other magnetic storage devices, or any other medium that can be used to carry or store desired program code means in the form of instructions or data structures and that can be accessed by a general-purpose or special-purpose computer, or a general-purpose or special-purpose processor.

It is further noted that the present invention is not limited to the specific feature combinations expressly listed herein, which are only understood as examples. Other features and/or feature combinations may also be possible.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The following FIGURE is provided to support the understanding of the present invention:

FIG. 1 illustrates a schematic drawing of an exemplary dialogue-based medical decision system.

### DETAILED DESCRIPTION

In the following, the present disclosure will be more fully described hereinafter with reference to the accompanying FIGURE. However, the present invention may be embodied in different forms and should not be construed as limited to the embodiments set forth herein. Rather, these embodiments are provided so that this disclosure will be thorough and will convey the scope of the present invention to persons skilled in the art.

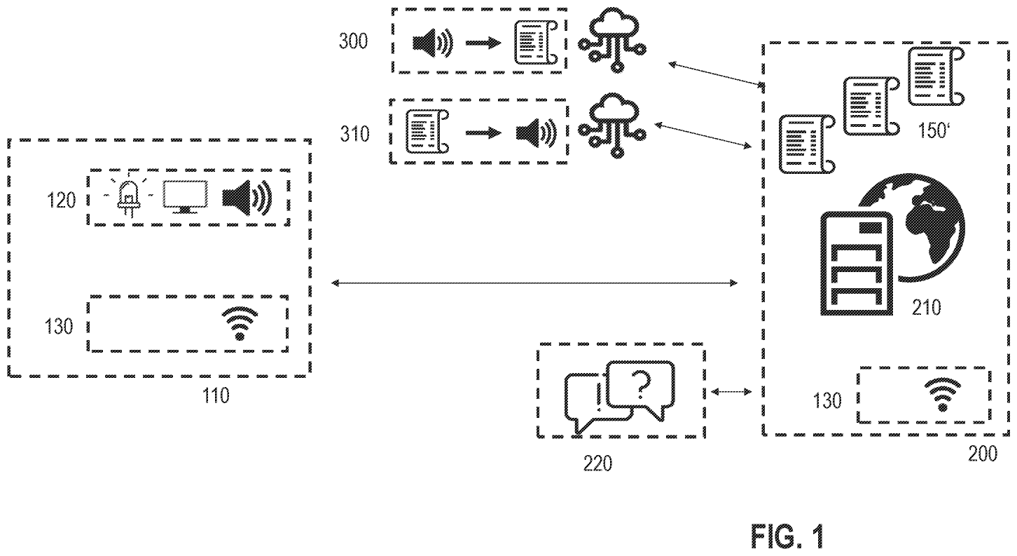

FIG. 1 shows an exemplary interaction of a patient device 110 with a dialogue-based medical decision system 200. In other examples, system 200 may interact additionally or alternatively with other (client) devices associated with the patient, e.g., devices of a doctor of the patient and/or a relative of the patient.

Patient device 110 may be a proprietary device specifically designated for the purposes herein, that may e.g., be in communication with an implant of a patient. In other examples, it may be a smartphone and or a smart home device having corresponding software functionality stored thereon to implement the aspects described herein (e.g., an app, and/or a skill). Patient device 110 comprises at least one (human-machine) interface 120 to interact with the patient. In the preferred embodiment, as shown in FIG. 1, the at least one interface 120 may at least comprise one of an LED flashlight, a display, a microphone and/or a loudspeaker. In addition the patient device 110 may also comprise a transmitter configured to transmit data associated with a health state of the patient to a dialogue-based medical decision system 200 and a receiver configured to receive messages, from the dialogue-based medical decision system 200. The transmitter and receiver may be implemented by an interface or means for communicating 130 with the dialogue-based medical decision system 200.

The dialogue-based medical decision system 200 may be accessible from all over the world. It may comprise at least one data processing unit 210. Furthermore, the dialogue-based medical decision system 200 may also comprise a transmitter and receiver for communicating with the patient device 110 and/or with at least one other device (e.g., as outlined above), e.g., via the internet or any other network system. Moreover, the same transmitter and receiver may be implemented to allow communication with at least one (other), e.g., cloud-based, service. The at least one other service may e.g., refer to a message-to-audio converter 310. It may be possible that a second service relates to an audio-to-message converter 300. The dialogue-based medical decision system 200 may still further be in communication with a dialogue system 220. The communication between the dialogue-based medical decision system 200 and the audio-to-message converter 300 and/or the message-to-audio converter 310 and/or the dialogue system 220 may be based at least in part on an internet and/or network system.

The means for communicating 130 of the dialogue-based medical decision system 200 may generally be similar to the communication means 130 of the patient device 110. In some examples, it may also differ, in that the patient device 110 is foreseen for wireless communication, whereas the system 200 may be wired, e.g., within the internet or a local network.

Communication between system 200 and patient device 110 is generally carried out via a secure connection between the respective means for communication 130, e.g., within or with a scalable IoT infrastructure. It may be direct or indirect with intermediate, e.g., cloud-based, infrastructures. Due to regional regulatory provisions, the system 200 may be operated in a decentralized manner by means of scalable infrastructures (cloud services, SaaS, etc.). Hence, it is possible that a patient device 110 in China communicate with a locally operated system 200, but devices 110 in other countries communicate with a system 200 operated outside of China.

In an example, the dialogue-based medical decision system 200 may initiate a dialogue with the patient, e.g., via patient device 110 (a dialogue may refer to at least one question provided to the patient and at least one corresponding reply from the patient).

The question may be obtained by the system 200 from the dialogue system 220. The dialogue system 220 may store a set of different and predefined dialogues, wherein each of the stored dialogues may comprise at least one message, e.g., a question. The predefined dialogues may be associated with different health situations of the patient. For example, there may be a predefined dialogue associated with problems with an implant of the patient. Moreover, the dialogue system 220 may also comprise a predefined dialogue associated with negative effects of a medication (e.g., side effects). A dialogue may generally comprise at least one question to be provided to the patient, a set of potential replies from the patient, possible follow up questions etc. (essentially allowing a decision tree, as outlined herein). Depending on the present health state of the patient, the dialogue-based medical decision system 200 may load a context-specific dialogue from the dialogue system 220, which comprises at least one (stored) message 150′. The processing unit 210 may select a message out of the messages 150′ and provide it to the patient. Depending on the received data from the patient device 110 (e.g., in response to a question in the selected message), the system may determine an updated current health state and/or provide the patient with one or more follow up questions. The selection of questions may additionally be supported by an artificial intelligence being part of the processing unit 210. Additionally or alternatively, the updated health state may be provided to another device, such as a device of a doctor or a relative (not shown).

The processing unit 210 may optionally comprise an input signal conditioning (filtering, scaling, norming, transforming, etc.) and/or an output post-processing (clustering, weighting, filtering, plausibility check, etc.).

The initiation of a dialogue with the patient device 110 may be triggered by the dialogue-based medical decision system 200. The selected at least one message of the stored messages 150′ may be in a text format. Prior to sending the selected message to the patient device 110, the dialogue-based medical decision system 200 may forward the selected message to the message-to-audio converter 310 which may be implemented as a cloud-based service, e.g., on a remote server. In other examples, the cloud-based service may either be implemented on the same server (or maybe even part) of the dialogue-based medical decision system 200. The message-to-audio converter 310 may convert the selected message into an audio file. The data processing unit 210 of the dialogue-based medical decision system 200 may then forward the audio file to the communication means 130 of the dialogue-based medical decision system 200. As a result, the message will be transmitted, in an audio format, to the patient device 110. As outlined above, the patient device 110 may at least comprise one interface for outputting the received audio file. The interface outputting the received audio file may e.g., be a loudspeaker. In response to outputting the received audio file to the patient the patient may reply to the received question. In order to record the reply of the patient, the patient device 110 may additionally comprise means for receiving an input of the patient. Such means for receiving an input of the patient may e.g., refer to the microphone (or other means as outlined herein). For example, the patient device 110 may record a spoken sequence of the patient in response to the received question from the dialogue-based medical decision system 200. It may be possible that the patient device 110 transmits data to the dialogue-based medical decision system 200 that includes the reply of the patient, e.g., in an audio format, e.g., an audio file. The means for communication 130 of the dialogue-based medical decision system 200 receives the audio file and may forward it to the data processing unit 210 of the dialogue-based medical decision system 200. Since the dialogue-based medical decision system 200 preferably processes questions and associated replies in text format, the dialogue-based medical decision system 200 may forward the reply of the patient to the audio-to-message converter 300 (that may be implemented similarly as the message-to-audio converter 310).

As outlined above, the audio-to-message converter 300 may essentially convert the audio data, received from the patient, into a (storable) text message. The audio to message converter 300 may then return the message (in text format) back to the dialogue-based medical decision system 200. The message may then be used to characterize the current health state of the patient by means of the processing unit 210.

Based on the received message (data) associated with the patient, the processing unit 210, which may be implemented as an artificial intelligence, may update the current health state of the patient, and optionally provide it to a device of a doctor and/or relative. Also, it may select a message out of the at least one stored message 150′ of the loaded dialogue, e.g., as a follow-up question (which is in conjunction to the received message from the patient) to be provided to the patient. If the current health state indicates a significant change, also another dialogue may be loaded, from which a suitable message to be provided to the patient next, may be selected. The described sequence may be performed iteratively, i.e., follow-up messages may be selected such that a satisfying characterization of the health state and a satisfying consultation of the patient is facilitated. If it is determined that the health state of the patient has sufficiently been characterized, the patient may be provided with feedback as outlined herein, and the dialogue-based medical decision system 200 may terminate its activity.

In addition or alternatively, the dialogue-based medical decision system 200 may determine, by means of the artificial intelligence (implemented in the processing unit 210, whether the patient is at risk based at least in part on the received message from the patient device 110 (and thus the patient). The determination whether the patient is at risk may preferably be carried out by means of an artificial intelligence. The artificial intelligence may preferably be implemented as part of the processing unit 210. If it is determined that the patient is at risk, the dialogue-based medical decision system 200 may select a respective message and transmit the message to the patient and/or a relative and/or a doctor to indicate that the patient is currently at risk.

For example, for recognizing decompensation of a patient or a change of behavior, training with position information (e.g., GPS) and weather and calendar dates is important. For example, in a neuronal pain therapy, a patient moves significantly less, if pain is not adequately treated anymore or if pain increases. Another reason may however be that it is cold outside and/or there is a low pressure zone at the patient, and the patient moves less for these reasons. As a result of the training, a dialogue system 220 and a post-processing system may be provided. The dialogue system 220 may be operated within system 200 and/or in a decentralized manner (SaaS, cloud based). The post-processing system may implement the functionality as described herein, which may create further processes, such as informing a doctor and/or relative and/or another medical decision system 200 and/or an electronic health file management system, depending on the result of a dialogue with the patient. For example, such informing may occur, if the patient informs the system about strong pain and/or if the system has determined that the patient is decompensated. Training may also result in an adaptation of the frequency with which a dialogue is initiated.

It is further noted that it may also be possible that the selected message is not converted into an audio file. Alternatively and/or additionally it may also be possible that the dialogue-based medical decision system 200 directly transmits the selected message to the patient device 110. In such an application scenario the patient device 110 may essentially output the received message as, e.g., a push notification on the display of the patient device (i.e., by means of the interface 120), if the patient device is implemented as e.g., a smartphone.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

In the following, a potential application scenario of the dialogue-based medical decision system 200 and a patient (i.e., the patient device 110) is given:

1. The patient starts the dialogue:
110: “Please call my doctor. I feel very bad at the moment. I feel pain in my chest.”
200: “I've just called the ambulance for you and I've forwarded your current GPS location. The ambulance will arrive in approximately five minutes. I have also already informed the ambulance about the implementation of the pacemaker last month and the associated therapy.”

2. The patient starts the dialogue:
110: “Please tell me more about the life with a neuro-implant.”
200: “Would you like to see a video or would you like to hear a blog?”
110: “I would like to see a video.”
200: “Okay, I will play a video for you. Additionally, I see you are working with rather big alternating current machines. I will provide you with additional information thereon.”

3. The cloud system would like to verify whether a compensation of the patient associated with a heart failure therapy is given:
200: “Hi Bob, how do you feel today?”
110: “I feel fine, thank you!”
200: “I see that you have hardly moved for five days, even though there's great weather in Hamburg.”
110: “I have heavy legs.”
200: “Do you have any other symptoms?”
110: “Yes, I'm experiencing breathing issues.”
200: “Okay, I have just made an appointment for you at your cardiologist, Mr. Heartbeat, tomorrow morning at 10:30 AM. I've also informed him about your health state.”

4. The cloud system would like to verify whether a compensation of the patient associated with a heart failure therapy is given:
200: “Hi Bob, how do you feel today?”
110: “I feel great, thank you.”
200: “But I see that you're moving a lot.”
110: “Yes, that's right, I feel great.”
200: “Do you have any symptoms?”
110: “No, not at all.”
200: “Thank you. It's good to hear that you are okay. I've just forwarded the information to your doctor.”

5. The cloud system would like to know whether the neuro spinal cord stimulation of the patient is successful:
200: “Hi Bob, how do you feel today?”
110: “I have severe pain in my right leg. None of my applications on my smart phone seems to work.”
200: “Okay, I will inform your doctor that he should have it checked.”

6. Child inquiries about general condition of father who has HF therapy:
110: “Please tell me how my father is doing”
200: “Based on the conversation last week and his other vital signs that I have, his general condition is satisfactory.”
110: “I spoke to him on the phone this morning. He complained of shortness of breath and dizziness.”
220: “Thanks for the information. I have made a note of that. I will call your father immediately and find out. I will let you and the cardiologist know the results right away.”

1. A dialogue-based medical decision system comprising:
a receiver for receiving data associated with a health state of the patient sent from a device associated with the patient;
a processing unit adapted to select a message associated with the health state of the patient based on the received data and based on stored patient data; and
a transmitter for transmitting the message to the device and/or to another device associated with the patient.

2. The dialogue-based medical decision system according to claim 1, wherein the transmitter is further adapted to transmit information, associated with the health state of the patient, to the device such that the device is enabled to base the sent data on the information.

3. The dialogue-based medical decision system according to claim 1, wherein the system is further adapted:
to receive the data in the form of audio and/or visual data; and/or
to transmit the message comprising audio and/or visual data.

4. The dialogue-based medical decision system according to claim 1, wherein the processing unit comprises an artificial intelligence.

5. The dialogue-based medical decision system according to claim 1, wherein the processing unit is adapted to select the message from a set of stored dialogues.

6. The dialogue-based medical decision system according to claim 1, wherein the system is configured to transmit the message to a message-to-audio-converter prior to transmitting the message to the device and/or the another device.

7. The dialogue-based medical decision system according to claim 1, wherein the system is adapted to further transmit information to enable an output of the message by the device and/or by the another device as an audio output and/or as a visual output.

8. The dialogue-based medical decision system according to claim 1, wherein the receiver and/or transmitter are adapted to communicate based on a low-latency communication system.

9. The dialogue-based medical decision system according to claim 1, wherein the system is adapted to be activated based at least in part on a request from the device and/or the another device and/or based on a predetermined schedule.

10. The dialogue-based medical decision system according to claim 1, wherein the system is automatically activated periodically and/or based on sensor data of one or more sensors associated with the patient.

11. A device associated with a patient, comprising:
a transmitter configured to transmit data associated with a health state of the patient to a dialogue-based medical decision system;
a receiver configured to receive, from the dialogue-based medical decision system, a message associated with the health state of the patient based on the transmitted data and based on patient data stored by the dialogue-based medical decision system; and
an interface adapted to indicate the received message to the patient.

12. A method implemented by a dialogue-based medical decision system:
receiving data associated with a health state of a patient from a device associated with the patient;
processing the data to select a message associated with the health state of the patient, based on the received data and based on stored patient data; and
transmitting the message to the device and/or another device associated with the patient.

13. The method according to claim 12, wherein the method steps are adapted to be executed at least two times to converse with the patient and/or a relative of the patient and/or a doctor of the patient.

14. A method implemented by a device associated with a patient:
transmitting data associated with a health state of a patient to a dialogue-based medical decision system;
receiving, from the dialogue-based medical decision system, a message associated with the health state of the patient based on the transmitted data and based on patient data stored by the dialogue-based medical decision system; and
indicating the received message to the patient.

15. A non-transitory computer-readable medium comprising a computer program, comprising instructions which, when executed, cause a computer to perform the steps of the method according claim 12.
